SEQUENCE LISTING (8 sequences)

SEQ ID NO: 1 - DNA, 21 bp, human
  primer (1)..(21)
gccatgaagtccctgtctctg

SEQ ID NO: 2 - DNA, 25 bp, human
  primer (1)..(25)
gggattaggagcagatgatagggta

SEQ ID NO: 3 - DNA, 28 bp, human
  primer (1)..(28)
aagagcatgcatgaagtccctgtctctg

SEQ ID NO: 4 - DNA, 39 bp, human
  primer (1)..(39)
aataagtcgaccagtgagtgccctcactctgctgctttc

SEQ ID NO: 5 - DNA, 20 bp, human
  primer (1)..(20)
gtgttactcatagcgcgtaa

SEQ ID NO: 6 - DNA, 21 bp, human
  primer (1)..(21)
ccatcaaacgagttggtgctc

SEQ ID NO: 7 - protein, 18 aa, rabbit
  mrna (1)..(18)
RGGCYRAMVEALAAPADC

SEQ ID NO: 8 - protein, 16 aa, rabbit
  mrna (1)..(16)
DGQDVYAHLVQQRLEA

(19) United States
(12) Patent Application Publication
(10) Pub. No.: US 2011/0117137 A1
(43) Pub. Date: May 19, 2011

(54) TAPASIN AUGMENTATION FOR ENHANCED IMMUNE RESPONSE

(75) Inventor: Wilfred Jefferies, Surrey (CA)

(73) Assignee: TAPIMMUNE, INC., Seattle, WA (US)

(21) Appl. No.: 12/864,751

(22) PCT Filed: Jan. 27, 2009
(86) PCT No.: PCT/IB09/05030

§ 371 Date: Jan. 31, 2011

Related U.S. Application Data
(60) Provisional application No. 61/024,004, filed on Jan. 28, 2008.

(51) Int. Cl. A61K 39/39; C12N 5/071; A61P 37/04; A61P 35/00; A61P 31/12; A61P 31/04; A61P 31/16; A61P 31/06; A61P 35/02

(52) U.S. Cl. 424/281.1; 424/278.1; 435/375

(57) ABSTRACT

Tapasin (Tpn) is a member of the MHC Class I loading complex and functions to bridge the TAP peptide transporter to MHC Class I molecules. Metastatic human carcinomas express low levels of the antigen processing components (APCs) tapasin and TAP, and display few functional surface MHC Class I molecules. As a result, carcinomas are often unrecognizable by effector cytolytic T cells (CTLs). Tpn alone can enhance survival and immunity of mammals against tumors, but additionally, Tpn and TAP can be used together as components of immunotherapeutic vaccine protocols to eradicate tumors.

### BACKGROUND OF THE INVENTION

The MHC Class I antigen presentation pathway is important both for the initiation of anti-tumor immune responses through cross-presentation of tumor antigens to CD8⁺ T cells, and in the recognition and killing of tumor cells by tumor-specific cytotoxic lymphocytes (CTLs). An important component in both these processes is the chaperone protein Tapasin (Tpn), a 48 kDa type I membrane glycoprotein whose function is assisting in the loading of antigenic peptides onto Class I molecules in the endo reticulum (ER). The mechanisms by which Tpn mediates this function include retaining empty MHC Class I molecules in the ER until loaded with peptides, stabilizing transported associated with antigen processing protein (TAP), bridging MHC Class I antigens to TAP, and supporting the binding of high-affinity peptides to MHC Class I antigen. In the presence of Tpn, surface MHC Class I molecules are more stable and thus more efficient at presenting antigens to CTLs or their precursors. Defects in Tpn expression lead to the destabilization of the MHC Class I loading complex including TAP1 and TAP2, and a reduction in the expression of MHC molecules at the cell surface.

Tpn is known to be down-regulated in many human carcinomas such as breast cancer, melanoma, colorectal carcinoma, and both small cell and non-small cell lung carcinoma, as well as mouse cancers such as mouse fibrosarcoma and mouse melanoma. Remarkably, in human colorectal cancers, Tpn is more frequently lost than TAP1, latent membrane protein 2 (LMP2) and latent membrane protein 7 (LMP7), suggesting that the loss of Tpn could be a key event in overcoming immune-surveillance in these tumors. Moreover, down-regulation or deficiency of components including Tpn in the MHC Class I antigen presentation pathway results in reduced immunogenicity of tumors and is associated with disease progression and disease outcome in a variety of human carcinomas. The mouse lung carcinoma cell line CMT.64, derived from a spontaneous lung carcinoma in a C57BL/6 mouse, is characterized by the down-regulation of many components of the antigen presentation pathway, including MHC Class I heavy chain, β₂-microglobulin, LMP2 and LMP7, TAP1 and TAP2, and Tpn. A number of studies have demonstrated that the restoration of TAP-1 expression in CMT.64 and other tumor cells using replicating vaccinia virus or non-replicating adenovirus increases the tumor antigen-specific immune responses and prolongs animal survival.

Accordingly, it is an objective of this invention to determine whether human Tpn (hTpn), either alone or in combination with human TAP1 (hTAP1), expressed from non-replicating adenoviruses, can restore antigen presentation, increase tumor antigen-specific immune responses, and prolong the survival of tumor-bearing mammals.

### SUMMARY OF THE INVENTION

This invention is directed to the expression of Tpn in Tpn-deficient cancer cells to restore the expression of functional surface MHC Class I antigen complexes, augment tumor cell immunogenicity and promote long term survival of animals bearing these metastatic tumors. The expression of Tpn in the Tpn-deficient mouse hepatoma cell line H6 carcinoma cell line and the human HepG2 cell line has been shown to increase surface MHC Class I expression, suggesting that this approach can be effective in treating many carcinomas. The results presented here indicate that the enhanced MHC Class I surface expression and immunogenicity due to AdhTpn infection in vivo significantly retards CMT.64 tumor growth and enhances animal survival. It is believed that the AdhTpn injections localized to the site of the tumor infect the CMT.64 cells and increase the activity of the endogenous antigen presentation pathway, leading to surface expression of MHC Class I-restricted tumor antigens that can then be recognized by the increased numbers of tumor-infiltrating CD8⁺ T cells, assisted by CD4⁺ T cells and CD11c⁺ dendritic cells (DCs).

The restoration of surface MHC Class I expression and increased immunogenicity of the tumor cells occurs despite multiple APC defects in CMT.64 cells, which include the down-regulation of MHC Class I heavy chain, β₂-microglobulin, TAP1, TAP2, LMP2, and LMP7. Residual transport of the peptides into the ER may be due to low levels of TAP expression (undetectable by Western blot) providing sufficient MHC Class I peptide complexes in the presence of Tpn-mediated chaperone activity for a significant increase in susceptibility to killing by specific T cell effectors. Steady state levels of other components of the antigen presentation pathway including TAP have been shown to be stabilized by Tpn. Therefore, Tpn expression in CMT.64 cells may stabilize the low level of TAP present in these cells, and therefore significantly increase the H-2K^{b} and H-2D^{b} surface expression and immunogenicity of CMT.64 cells in this manner. Combining AdhTAP1 and AdhTpn in treating carcinomas (which is deficient in both these components) results in enhanced protection and survival in tumor-bearing animals.

Adding to the novelty of these findings, this appears to be the first indication in which increased Tpn expression in mice increases antigen-specific immune responses to exogenously acquired antigens (OVA). Components of the peptide loading complex that are essential for direct antigen presentation by virus-infected cells or tumor cells to circulating CD8⁺ T cells are also required for indirect presentation by professional antigen-presenting cells to precursor CD8⁺ T cells during the initiation of tumor antigen-specific immune responses. The additional Tpn expression that increases cross-presentation activity of DCs in vitro could be a combination of Tpn and a vector effect, suggesting an interaction between the antigen presentation pathway and innate mechanisms. The ability of DCs from mice infected with AdhTpn in combination with OVA to activate SIINFEKL-specific B3Z cells demonstrates a physiologically relevant in vivo correlation of the effects seen in vitro. The increase in cross-priming activity in vivo due to AdhTpn infection was further demonstrated by increases in the number of SIINFEKL-specific CD8⁺ T cells in both peripheral blood and spleen as measured by tetramer staining.

The mechanism of increased cross-priming may correlate with the significant increase of CD4⁺ TILs within tumor masses of mice treated with AdhTpn, which could be related to immunogenicity of the adenovirus vector itself. Large numbers of CD4⁺ T cells favour the CD4⁺ T cell dependent pathway of CD8⁺ T cell activation, whereby the CD4⁺ T cells may stimulate DCs through CD40 ligand and/or present alternative signals that can license DCs for cross-priming, or directly stimulate CD8⁺ T cells by cytokines such as interleukin-2.

Adenoviral vectors containing the APC genes encoding Tpn and TAP1 can play an important role in future cancer immunotherapies. The restoration of Tpn together with TAP have several advantages over other existing approaches and provide a general method for increasing immune responses against tumors regardless of the antigenic composition of the tumor or the MHC haplotypes of the host.

### BRIEF DESCRIPTION OF THE DRAWINGS

FIG. 1 is a series of graphs and blots showing that Tapasin expression in CMT.64 cells after infection with AdhTpn is dose dependent and leads to increased surface MHC Class I levels and presentation of a viral epitope. FIG. 1A is a blot and graph showing CMT.64 cells infected with AdhTpn at an MOI of 1, 5, 25, 50, and 100 PFU/cell of AdhTpn or Ψ5 at 100 PFU/cell and harvested 48 h later. Western blotting was carried out with anti-hTpn, mTAP1, and mTAP1 polyclonal antibodies and β-actin mAb. β-actin was used as a control for protein loading. Densitrometry was performed on the hTpn bands to quantify the amount of protein produced by the AdhTpn infection at each dose. FIG. 1B are graphs showing that AdhTpn infection increases both H-2K^{b} and H-2D^{b} surface expression in CMT.64 cells. Ψ5-adenovirus vector control, IFN-γ-positive control. FIG. 1C shows that the infection of CMT.64 cells with AdhTpn restores MHC Class I antigen presentation of VSV-NP epitope and increases susceptibility to lysis by VSV-NP-specific effector cells. Targets: CMTNSV-NP-CMT.64 transfected with VSV-NP (52-59) minigene, CMTNSV-NP infected with T5 (adenovirus vector control), or CMT/VSV-NP infected with AdhTpn. Effectors: splenocytes from VSV-infected mice.

FIG. 2 is a series of graphs and photomicrographs showing that AdhTpn increases dendritic cell cross-priming of ovalbumin antigen. FIG. 2A shows that AdhTpn increases DC cross-presentation of OVA antigen in vitro. Splenic DCs were infected with AdhTpn or Ψ5 for 2 hrs followed by incubation with OVA for 16 hrs and then stained with 25.D1.16 and measured by FACS analysis. FIGS. 2B and 2C show that AdhTpn infection promotes cross-priming of CD8⁺ T cells after immunization with soluble OVA. C57BL/6 mice were i.p. injected with AdhTpn, or PBS; 16 hrs later, mice were injected s.c. with OVA and boosted with the same virus and OVA at day 7. After 8 d, splenic DCs were cultured at different ratios with B3Z T cells. After 24 hrs of co-culture, B3Z activation—assessed by β-Galactosidase production—was measured by ELISA plate reader. FIG. 2D shows the percentage of CD8⁺ T cells that recognize the ovalbumin-derived immunodominant peptide SIINFEKL on MHC Class I molecules of spleen and blood APCs were quantified by H-2K^{b}/SIINFEKL tetramer staining.

FIG. 3 are graphs showing that AdhTpn and AdhTAP1 prolong the survival of tumor-bearing mice. FIG. 3A shows that C57BL/6 mice were injected i.p. with CMT.64 cells (4×10⁵ cells/mouse) and were treated on days 1, 3, 5, and 8 with either AdhTAP1 at 1.25, 2.5, 5.0, 10×10⁷ PFU, ψ5 at 1×10⁸ PFU in 500 μl PBS, or PBS and survival was followed for 90 days. The lowest dose showing a protective effect (2.5×10⁷ PFU) was chosen for complementation studies with AdhTpn. FIG. 3B shows that treatment with AdhTpn, AdhTAP1, AdhTAP1 and AdhTpn, T5, (5.0×10⁷ PFU/500 μl PBS) or PBS was done as above and survival was followed for 90 days (n=10 mice per group). To ensure all groups received the same number of Ad particles, mice treated with AdhTAP1 alone or AdhTpn alone were complemented with an equal amount of ψ5 vector to maintain a total Ad dose of 5×10⁷ PFU. At the same dose, AdhTAP1 and AdhTpn together resulted in maximal protection that was stastically greater than AdhTAP1 alone and Ψ5 and PBS controls, but not AdhTpn alone (p=0.0061 for AdhTAP1+AdhTpn vs. AdhTAP1 alone). Survivorship of mice treated with AdhTAP1+AdhTpn (2.5×10⁷ PFU of each virus) was similar to that of mice treated with the highest dose (1×10⁸ PFU) of AdhTAP1 alone.

FIG. 4 are photomicrographs showing that tumor infiltrating lymphocytes and DCs were increased in CMT.64 tumors treated with AdhTpn in vivo. IHC staining for CD4⁺ (FIG. 4A), CD8⁺ (FIG. 4B) or CD11c⁺ (FIG. 4C) cells in CMT.64 tumors treated with AdhTpn (A, D, G) or Ψ5 (Ad vector control), or PBS. Tumors were analyzed 19 days after CMT.64 cells were introduced into mice. C57BL/6 mice were injected i.p. with CMT.64 cells (4×10⁵ cells/mouse) and were treated on days 1, 3, 5, and 8 with either 2.5×10⁷ PFU/mouse of AdhTpn or Ψ5 or PBS only. A positive stain is indicated by the intense brown labelling of cell surface membranes (200× magnification).

FIG. 5 is a graph showing that tumor infiltrating lymphocytes were increased in CMT.64 tumors treated with AdhTpn in vivo by FACS analysis (**p=0.011 for CD8 in treated vs. PBS control. *p=0.042 for CD4 in treated vs. PBS control after a square root transformation to satisfy homogeneity of variance). Tumor infiltrating CD4⁺ and CD8⁺ lymphocytes are presented as a percentage of total cells in tumors.

### DETAILED DESCRIPTION OF THE INVENTION

The invention will now be further described in the following detailed examples, which are presented as illustrative only, and should not be construed to otherwise limit the scope or spirit of the invention or any of its embodiments.

### Materials and Methods

### Cells, Viruses, and Mice

HEK 293 cells (ATCC, Rockville, Md., U.S.A.), CRE8 cells (S. Hardy et al.; J. Virol; 71: 1842-1849 (1997)), CMT.64 cells (Y. Lou et al; Cancer Res.; 65: 7926-7933 (2005); CMT/VSV-NP (CMT.64 transfected with VSV nucleocapsid protein (NP) minigene containing the immunodominant epitope from amino acids 52 to 59 presented on H-2K^{b}) and T1 (ATCC, CRL-1991, a hTpn positive cell line) were cultured in Dulbecco's modified Eagle medium supplemented with 10% FBS. CRE8 cells have a 13-actin-based expression cassette driving a Cre recombinase gene with an N-terminal nuclear localization signal stably integrated into HEK 293 cells (S. Hardy et al., supra). Ψ5 virus is an E1 and E3 deleted version of Ad5 containing loxP sites flanking the packaging site (S. Hardy et al., supra). Ψ5 and recombinant adenovirus were propagated and titred in HEK 293 cells. Primary mouse splenocytes and 0.220 cells (Tpn-deficient human myeloma cells, provided by Dr. Peter Cresswell, Yale University School of Medicine, New Haven, Conn., U.S.A.) were cultured in complete culture medium consisting of RPMI 1640+10% FBS. Six to eight week old C57BL/6 (H-2^{b}) female mice were obtained from The Jackson Laboratory (BarHarbor, Me., U.S.A.) and housed at the Biotechnology Breeding Facility, University of British Columbia, under Canadian Council on Animal Care guidelines.

### Construction of Non-replicating Adenovirus/Human Tapasin (AdhTpn)

FirstChoice™ Total RNA from human spleen was obtained from Ambion Inc. (Austin, Tex.). cDNA was synthesized using RETROscript® First strand synthesis kit for RT-PCR (Ambion Inc.) using Oligo(dT) primers as per the manufacturer's instructions. Tpn cDNA was amplified using primers designed based on the sequence of human Tpn transcript variant 1 (NM_{—}003190) using Pfu DNA polymerase (Stratagene, La Jolla, Calif.). The primer sequences used were as follows: forward primer 5′-GCCATGAAGTCCCTGTCTCTG-3′ (SEQ ID NO:1) and reverse primer 5′-GGGATTAGGAGCAGATGATAGGGTA-3′ (SEQ ID NO:2). The insert was cloned in pCR-Blunt II-TOPO vector (Invitrogen Life Technologies, Carlsbad, Calif.) and both strands were sequenced to ensure no mutations were present. HTpn was digested from TOPO/hTpn with Pst I ad BamHI and then cloned into a Pst I- and BamHI-digested shuttle vector, padlox plasmid (S. Hardy et al., supra). The resulting vector, Pad/hTpn, was isolated and sequenced to ensure the sequence fidelity. The AdhTpn was generated as previously described (S. Hardy et al., supra). Briefly, the pad/hTpn, linearized with SfiI, was co-transfected along with Ψ5 DNA into CRE8 cells using LipofectAMINE PLUS™ Reagent (Invitrogen Life Technologies) to generate AdhTpn. AdhTpn recombinant viral clones were identified by immunofluorescence assay and plaque purified three times in HEK 293 cells. The recombinant virus was amplified in large-scale stock in HEK 293 cells, purified by CsCl density gradient centrifugation, and titred in HEK 293 cells. The identity of AdhTpn was confirmed by PCR and DNA sequencing of purified viral DNA using primers specific for Tpn and adenovirus DNA flanking either side of the Tpn gene. The primer sequences were as follows: forward primer 5′-AAG AGC ATG CAT GAA GTC CCT GTC TCT G-3′ (SEQ ID NO:3) and reverse primer 5′-AAT AAG TCG ACC AGT GAG TGC CCT CAC TCT GCT GCT TTC-3′ (SEQ ID NO:4) for amplification of Tpn; forward primer 5′-GTG TTA CTC ATA GCG CGT AA-3′(SEQ ID NO:5) and reverse primer 5′-CCA TCA AAC GAG TTG GTG CTC-3′ (SEQ ID NO:6) for amplification of adenoviral flanking sequence.

### TAP and Tpn Expression After AdhTpn Infection of CMT.64 Cells

To examine Tpn and TAP expression in response to increasing doses of AdhTpn, CMT.64 cells were infected with AdhTpn at 1, 5, 25, 50, and 100 PFU/cell or Ψ5 (negative control) at 100 PFU/cell. T1 cells and 0.220 cells were, respectively, used as hTpn positive and negative controls. CMT.64 cells treated with IFN-γ were a positive control for mouse TAP1 (mTAP1), mouse TAP2 (mTAP2) and mouse Tpn (mTpn) expression. Two days after infection, cells were lysed and subjected to SDS-PAGE and electro-transferred to Hybond PVDF membrane (Amersham Biosciences, Buckinghamshire, England). The blot was treated with rabbit anti-hTpn antibodies (StressGen Biotechnologies Corp, Victoria, BC, Canada), rabbit anti-mTpn antibodies (a gift from Dr. David Williams, University of Toronto), rabbit anti-mTAP1 and rabbit anti-mTAP2 (made by our Lab by immunizing rabbits with synthetic peptides generated from the mTAP-1 (RGGCYRAMVEALAAPAD-C) (SEQ ID NO:7) or mTAP-2 (DGQDVYAHLVQQRLEA) (SEQ ID NO:8) a peptide corresponding to the last 16 amino acids at C-terminal end of mouse TAP2) sequences conjugated to KLH (Q. J. Zhang, Int. J. Cancer (2007)), and mouse monoclonal antibody (mAb) against human β-actin (Sigma-Aldrich Oakville, ON, Canada). Goat anti-rabbit IgG (H+L)-HRP and goat anti-mouse IgG (H+L)-HRP (Jackson ImmunoResearch Lab, West Grove, Pa.) were used as secondary antibodies. The bands were visualized by enhanced chemiluminescence and exposure to Hyperfilm (Amersham Biosciences). Line densitometry was performed using the AlphaEaseFC software, version 6.0.0 (Alpha Innotech, San Leandro, Calif.).

### Effect of AdhTpn on Surface Expression of MHC Class I

CMT.64 cells were infected with AdhTpn or Ψ5 at 50 PFU/cell. Two days after infection, the cells were incubated with anti-MHC class I mAbs, y3 (H-2K^{b}-specific) and 28.14.8S (H-2D^{b}-specific), at 4° C. for 30 min. Bound antibodies were detected by goat anti-mouse IgG-FITC (Jackson ImmunoResearch Lab). The FACS analysis was performed in a FACSCaliburTM® (Becton Dickinson, Franklin Lakes, N.J.).

### CTL Assay

Cytotoxicity was measured in a standard 4 hr ⁵¹Cr-release assay. In brief, stably-transfected CMT.64 cells (CMT/VSV-NP) expressing the vesicular stomatitis virus nucleoprotein (VSV-NP) which contains an immunodominant viral peptide consisting of amino acids 52-59 were infected with AdhTpn or Ψ5 at 50 PFU/cell for 1 day. These cells were labelled with Na₂⁵¹CrO₄ (Amersham Biosciences) and used as targets for VSV-specific effector cells. VSV-specific CTL effectors were generated by i.p. injection of 5×10⁷ PFU of VSV into mice. Splenocytes were collected five days after infection and cultured in RPMI-1640 complete medium plus 1 μM VSV-NP (52-59) peptide for five days.

### In Vitro Cross-Presentation of Ovalbumin by DCs

Spleens were obtained from C57BL/6 mice as described (and disrupted by injection of 1 ml RPMI-1640 medium containing 5% FCS, 1 mg Collagenase D (Roche Applied Science, Laval, Qc, Canada) and incubated for 30 min at 37° C. Subsequently, DC-enriched cell populations were obtained by centrifugation of cell suspension on Ficoll-Paque (Amersham Biosciences) gradients. DCs were then purified by positive selection with anti-CD11c MACS beads (Miltenyi Biotech, Auburn, Calif.) with the resulting population being >98% CD11c⁺. Splenic DCs were then infected with either AdhTpn or Ψ5 at 20 PFU/cell for 2 hrs followed by incubation with ovalbumin (OVA) (Worthington Biochemical Corporation, Lakewood, N.J.) at 5 mg/ml for 16 hrs at 37° C. DCs were washed and Fc receptors blocked with 2.4G2 FcγIII/II blocker (BD PharMingen, Mississauga ON, Canada) before staining with 25.D1.16 mAb (A. Porgador, Immunity, 6:715-726 (1997), specific for H-2K^{b}/SIINFEKL, followed by phycoerythrin (PE)-conjugated rat anti-mouse IgG1 antibody (Jackson ImmunoResearch Lab.). Flow cytometry was used to quantify H-2K^{b}/SIINFEKL complexes on surface of DCs.

### In Vivo Cross-Presentation of Ovalbumin and Generation of Specific Immune Responses

On day 0, mice were infected i.p. with 1×10⁸ PFU AdhTpn, Ψ5, or PBS. Soluble OVA (30 mg in 100 μl) was injected s.c. 16 hrs later and the animals were boosted with the same dose of virus and OVA at day 7. To study the cross-priming activity of DCs, splenic DCs were isolated from mouse spleens 24 hrs later, fixed in 0.005% glutaraldehyde and cultured at 37° C. in a 96-well plate in the presence of different ratios of B3Z (an IL-2-secreting, LacZ-inducible T cell hybridoma that can be activated upon recognition of H-2K^{b}/SIINFEKL complexes (N. Shastri, J. Immunol, 150: 2724-2736 (1993)),—a gift from Dr. Nilabh Shastri, University of California Berkeley, Calif. Following 24 hrs of co-culture, activation was measured by assessing the β-galactosidase production following addition of Chlorophenol Red-B-D-Galactopyranoside (CPRG, Roche Applied Science). The plate was read on ELISA plate reader 24 hrs later at 595 nm with the 630 nm background absorbance subtracted. On day 5 following the last immunization, venous blood was collected and enriched lymphocyte populations were obtained by centrifugation of blood on Ficoll-Paque gradient. Spleens were also harvested, digested as described above and splenocyte-enriched populations were generated in the same fashion. Lymphocytes and splenocytes were double stained with iTAg™ H-2K^{b}/SIINFEKL-PE (Beckman Coulter Canada Inc, Mississauga, ON, Canada) and anti-CD8-FITC (Ly-2) (BD PharMingen) antibodies to determine total and CD8⁺ splenocytes specific for H-2K^{b}/SIINFEKL. FACSCalibur™ was used to collect the data which were analyzed using FlowJo software.

Treatment of CMT.64 Tumor-Bearing Mice with AdhTpn and AdhTAP1

For titration of the virus dose, tumors were established in six groups of 3 or 4 mice per group by i.p. injection of 4×10⁵ CMT.64 cells in 5000 PBS. On day 1, 3, 5, 8 days after the introduction of CMT.64 cells, the mice were further i.p. injected with either AdhTAP1 at 1.25, 2.5, 5.0, 10×10⁷ PFU, ψ5 at 1×10⁸ PFU in 500 μl PBS, or PBS and survival was followed for 90 days. For AdhTpn or AdhTpn plus AdhTAP1 treatment in CMT.64 tumor-bearing mice, tumors were established in five groups of 14 to 18 mice per group by i.p. injection of CMT.64 cells (4×10⁵ cells in 5000 PBS). On 1, 3, 5, and 8 days after the introduction of CMT.64 cells, the mice were further i.p. injected with AdhTpn, AdhTAP1, AdhTAP1 and AdhTpn, T5, (5.0×10⁷ PFU/500 μl PBS.) or PBS and survival was followed for 90 days. To ensure all injection groups received the same number of Ad particles, mice treated with only one type of recombinant were complemented with enough ψ5 vector to maintain a total Ad dose of 5×10⁷ PFU. During the experiment four to eight mice of AdhTpn, ψ5 or PBS groups were sacrificed from each group at selected times to observe tumor growth patterns and to measure the number of tumor-infiltrating CD4⁺ and CD8⁺ T lymphocytes and CD11c⁺ DCs.

### Tumor Infiltrating Lymphocytes (TILs) and DCs

TILs and tumor-infiltrating DCs were analysed using both FACS and immunohistochemistry staining (IHC). Tumors were disaggregated into single cells and incubated with rat anti-mouse CD8 (Ly-2) mAb and R-PE-conjugated rat anti-mouse CD4 (L3T4) mAb, and the number of CD8⁺ and CD4⁺ TILs was quantified by FACS. Acetone fixed cryosections (8 μm) of frozen tumors were stained for tumor infiltrating cells (CD8⁺, CD4⁺ T cells, and CD11c⁺DCs) with rat anti-mouse CD4 mAb (RM4-5), rat anti-mouse CD8 mAb (53-6.7), or hamster anti-mouse CD11c (HL3). Rat IgG₂ₐ was used as isotope control for anti-CD8 and anti CD4 antibodies, whereas hamster IgG was the control for the antibody detecting CD11c⁺ cells. Antibody binding was detected with biotinylated polyclonal anti-rat IgGs and biotinylated anti-hamster IgG secondary antibodies and streptavidin-HRP and a DAB detection system (all the reagents were purchased from BD Biosciences PharMingen).

### Statistical Analysis

For the cross-presentation assays, the Chi Squared Test (Multivariate Comparison, FlowJo 3.7.1.) was used to analyze FACS histograms for differences in total H-2K^{b} or H-2K^{b}/OVA₂₅₇₋₂₆₇ complexes expressed on DCs infected AdhTpn or Ψ5 (control vector) following incubation with OVA. Results were considered significant if p<0.01 (99% confidence), and T(X)>10 was empirically determined as a cut off value. Histograms representative of one of four repeated experiments have been shown. Survivorship data was analyzed using the “Comparison of survival distributions” methodology. The data were considered statistically different if p<0.05.

### Results

### AdhTpn Increases MHC Class I Surface Expression and Immunogenicity in CMT.64 Cells.

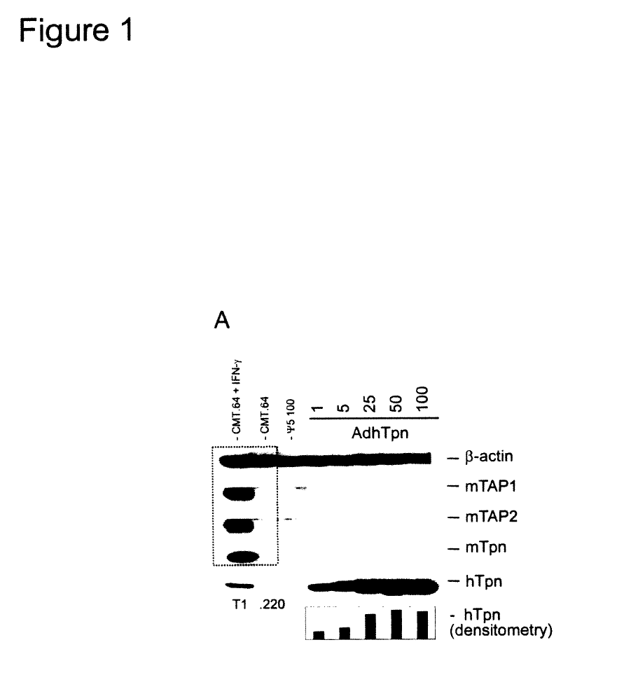

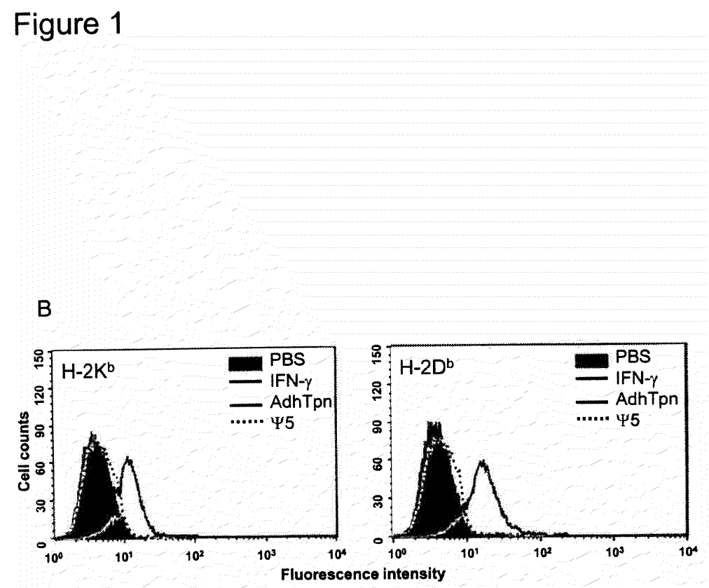

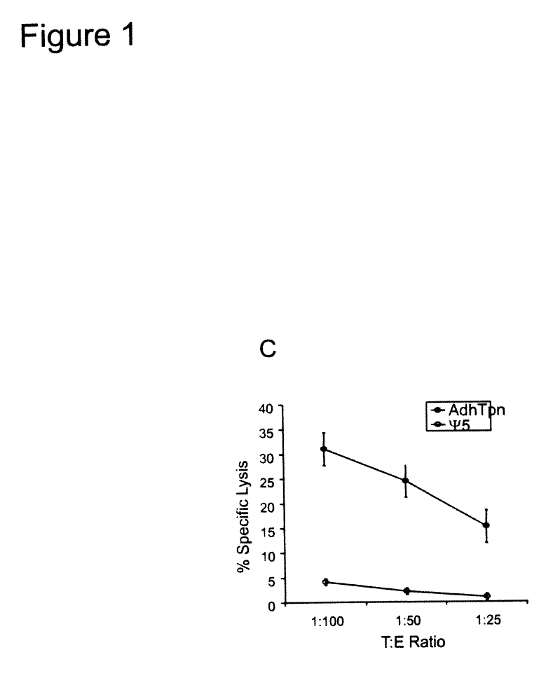

CMT.64 cells infected with AdhTpn expressed hTpn in a dose dependent manner (FIG. 1A). However, no increase in endogenous mTpn, mTAP1 and mTAP2 protein expression was detected in AdhTpn-infected CMT.64 cells by Western blot. Nevertheless, flow cytometry analysis showed that cell surface expression of H-2K^{b} and R-2D^{b} was increased in CMT.64 cells infected with AdhTpn (FIG. 1B), whereas cells infected with Ψ5 showed no such increase. CMT.64 cells treated with IFN-γ were used as a positive control and showed much larger increases in H-2K^{b} and H-2D^{b} surface expression (FIG. 1B), as well as increases in endogenous mTpn, mTAP 1 and mTAP2 protein levels in Western blot analysis (FIG. 1A). AdhTpn also enhanced the ability of CMT.64 stably transfected with the VSV nucleoprotein minigene (CMT/VSV-NP) to present the immunodominant VSV-NP₅₂₋₅₉ peptide to CTLs. CMT/VSV-NP cells infected with AdhTpn were sensitive to the cytolytic activity of VSV-specific effector T lymphocytes, while CMT/VSV-NP cells alone or infected with Ψ5 were resistant to killing (FIG. 1C), presumably due to the lack of H-2K^{b}/VSV peptide on the cell surface of the latter cells. These results show that hTpn expression and activity following AdhTpn infection can restore sufficient MHC class I-restricted antigen presentation of a specific epitope (VSV-NP₅₂₋₅₉) to make these cells susceptible to specific CTL activity.

### AdhTpn Increases Dendritic Cell Cross-Presentation and Cross-Priming

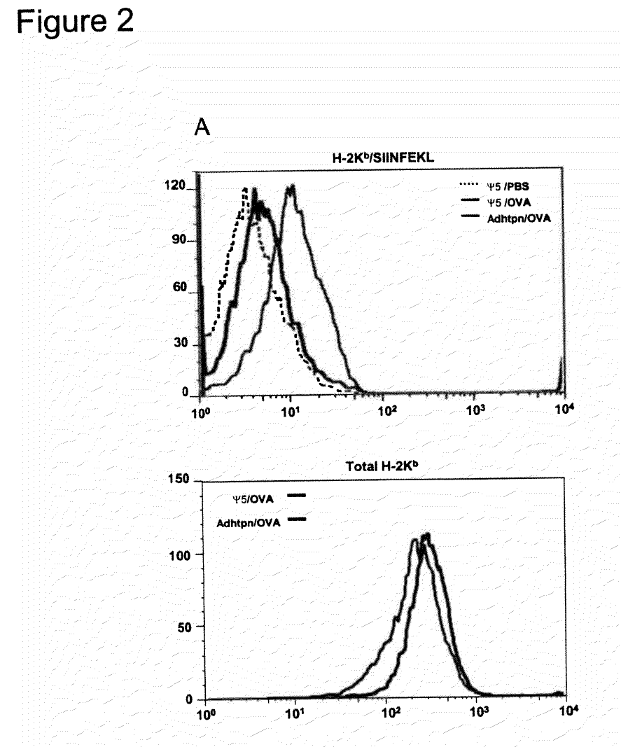

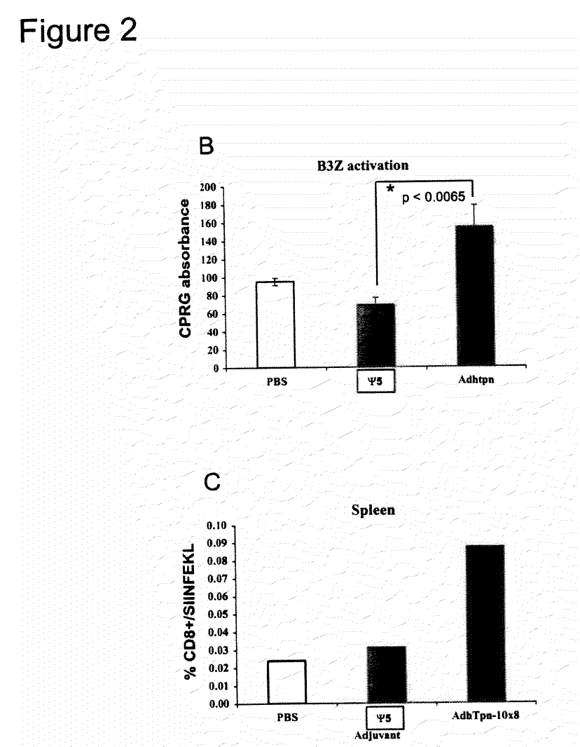

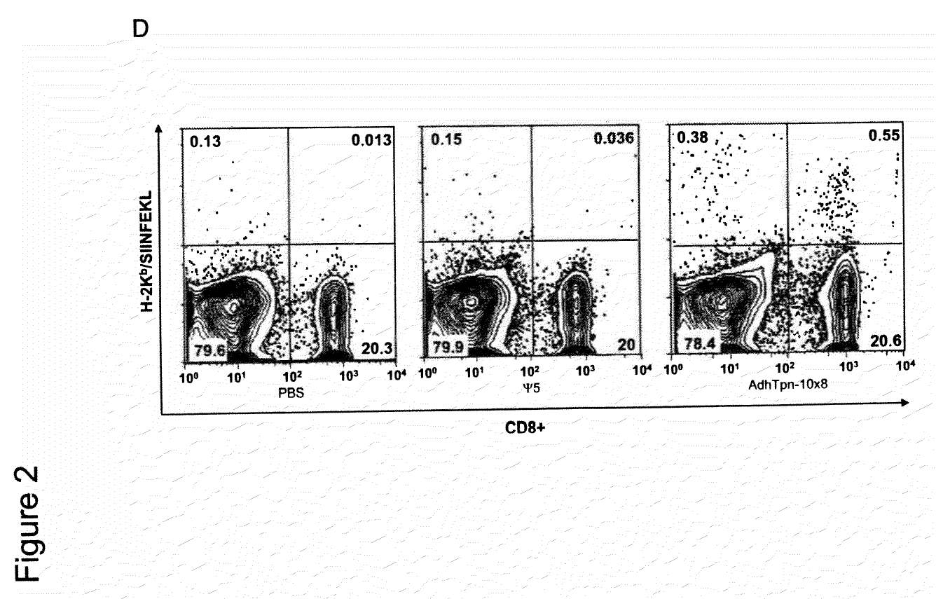

The model antigen OVA was used to assess the ability of DCs infected with AdhTpn to cross-present the immunodominant peptide SIINFEKL in the context of H-2K^{b}. Flow cytometry provides a semi-quantitative readout of the number of cell surface H-2K^{b}/SIINFEKL complexes, allowing assessment of cross-presentation efficiency. Splenic CD11c⁺ DCs infected in vitro with AdhTpn showed significantly increased cross presentation of SIINFEKL on H-2K^{b} compared to DCs infected with ψ5 (p<0.01) (FIG. 2A). The total surface H-2K^{b} levels were also slightly increased in AdhTpn-infected DCs compared to Ψ5-infected DCs. To examine this effect in vivo, we administered TS, PBS, or AdhTpn i.p. and injected OVA subcutaneously in order to test the effect of AdhTpn in the generation of H-2K^{b}/SIINFEKL-specific CD8⁺ T cells. Spleen-derived DCs taken ex vivo from mice infected with AdhTpn and immunized with OVA had a greater capacity to activate the H-2K^{b}/SIINFEKL-specific T cell hybridoma, B3Z, than DCs from mice infected with vector alone (FIG. 2B). AdhTpn-infected mice immunized with OVA showed a greater general immune response, detected by an increased number of total CD8⁺ T cells (data not shown), and a significantly increased OVA-specific response, as shown by a greater number CD8⁺ T cells specific for H-2K^{b}/SIINFEKL (measured with tetramer staining) in the spleen compared to vector control (Ψ5) or PBS control. This increase in OVA-specific CD8⁺ T cells was even more prominent in peripheral blood from AdhTpn-infected mice compared to Ψ5 and PBS controls (FIG. 2C & FIG. 2D). This indicates that infection of splenic DCs with AdhTpn, but not Ψ5 alone, accounted for the increase in both general and antigen-specific CD8⁺ T cell responses, which in turn is likely due to increased cross-presentation of exogenous antigen in vivo.

### AdhTpn Treatment Increases Survival of Mice Bearing CMT.64 Tumors Better Than AdhTAPA Treatment, and Maximal Protection is Achieved by Combining Both AdhTpn and AdhTAP1

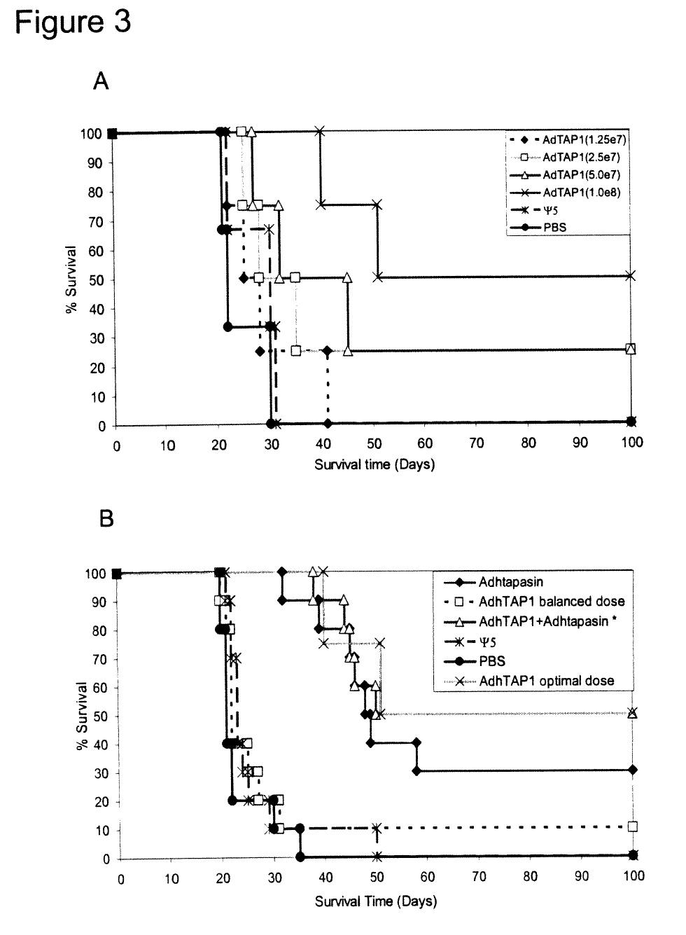

Previously, we demonstrated that treatment of CMT.64 tumor-bearing mice with recombinant adenovirus expressing human TAP1 (AdhTAP1) resulted in increased survival compared to mice treated with or PBS alone (Y. Lou et al., supra). Since AdhTpn increases MHC-I antigen surface expression and restores susceptibility to CTL killing in a manner similar to AdhTAP1 treatment, we examined if AdhTpn in combination with AdhTAP1 could enhance the inhibition of CMT.64 tumor formation. In order to avoid cytotoxicity associated with high adenoviral loads, a suboptimal dose of 2.5×10⁷ PFU of AdhTAP1 determined by titration (FIG. 3A) that was demonstrated to have a protective effect, was used in combination with an equal dose of AdhTpn. To balance the viral load, AdhTAP1 and AdhTpn alone treatments were mixed with an equal number of Ψ5 viruses. Dual treatment with AdhTpn and AdhTAP1 resulted in even greater mouse survival than either virus with Ψ5 alone, with 50% long-term survival without visible tumors (greater than 100 days) compared to 30% with AdhTpn and 10% with the low dose of AdhTAP1 (FIG. 3B). The dual treatment was statistically more effective than Ψ5 or AdhTAP1 treatment alone at the same viral dose (p<0.01), but not stastically different from AdhTpn treatment alone at the same dose. AdhTpn and AdhTAP1 at 2.5×10⁷ PFU of each virus (5×10⁷ PFU total virus) was equivalent to a much higher dose (1×10⁸ PFU) of AdhTAP1 alone, demonstrating that the dual treatment is more efficacious at a given dose (FIG. 3B).

### AdhTpn Treatment Increases TILs and Tumor-Infiltrating DCs in CMT.64 Tumors

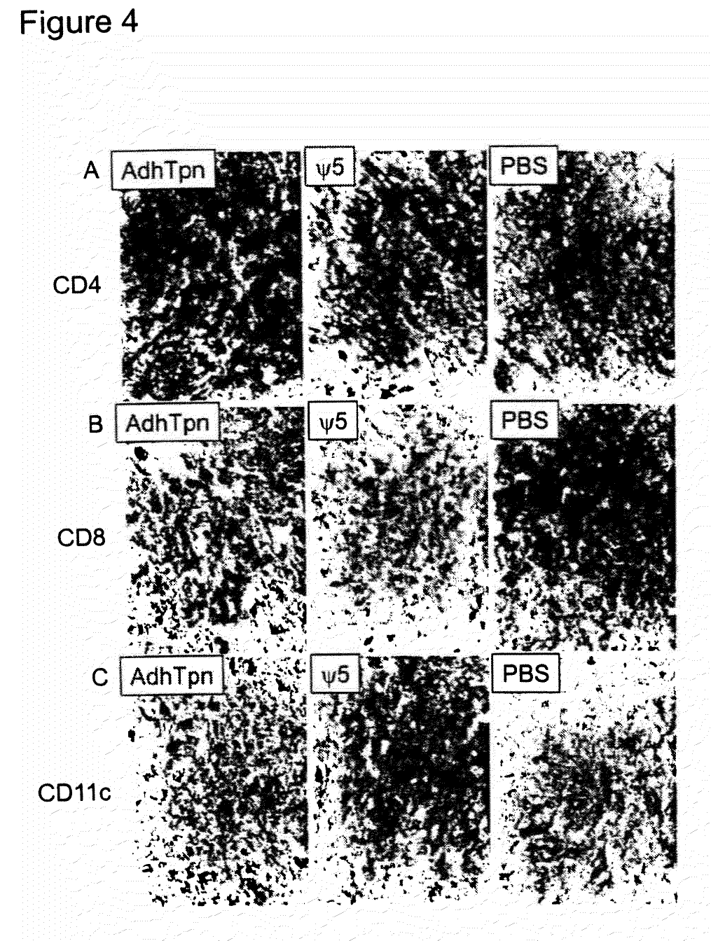

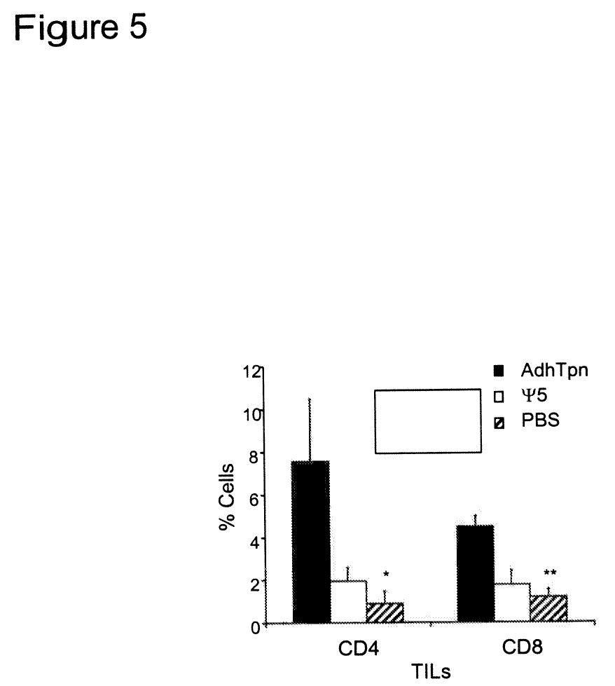

Between four to eight mice from the AdhTpn treatment group, as well as and PBS control groups, were examined for patterns in tumor growth 20 days after the last treatment injection. The peritoneal cavities of mice treated with AdhTpn were tumor-free or had only a few small tumors less than 1 or 2 millimeters in diameter. Both the liver and intestine appeared normal upon visual inspection. This was in sharp contrast to mice treated with PBS or Ψ5. These mice had large volumes of bloody ascites fluid (2-5 ml) and many tumors distributed throughout the peritoneal cavity. Tumors were observed growing on the liver and intestine and were associated with large fibrotic adhesions. Tumors harvested from the mice were examined for TILs and DCs infiltrates by FACS and IHC staining. IHC staining showed that mice treated with AdhTpn had significantly greater numbers of CD8⁺ and CD4⁺ T cells and CD11c⁺DCs in the tumor mass in tumors taken from mice treated with Ψ5 or PBS (FIG. 4). FACS analysis also confirmed that mice treated with AdhTpn had significantly greater CD8⁺ and CD4⁺ TILs (p=0.011 and p=0.042, respectively) than in tumors taken from mice treated with Ψ5 and PBS (FIG. 5). These results are consistent with our previous findings treating CMT.64 tumor-bearing mice with AdhTAP1 (10), and suggest that AdhTpn treatment may function in a similar manner by increasing tumor antigen-specific immune responses.

What is claimed is:

1. A process of enhancing an immune response to an antigen comprising administering, as the sole immune response enhancing agent, an effective amount of an agent that can augment the level of tapasin in a target cell bearing the antigen, to a cell or animal in need thereof.

2. The process of claim 1 wherein said agent comprises a nucleic acid encoding tapasin.

3. The process of claim 1 wherein the agent comprises a viral vector containing a nucleic acid encoding tapasin.

4. The process of claim 3 wherein the viral vector is an adenoviral vector.

5. The process of claim 1 wherein the agent comprises a plasmid vector containing a nucleic acid encoding tapasin.

6. The process of claim 1 wherein the target cell is a tumor cell.

7. The process of claim 1 wherein the target cell is a virally infected cell or a bacterial cell.

8. The process of claim 1 wherein said agent comprises tapasin.

9. The process of claim 1 wherein the animal is a human patient.

10. The process of claim 9 wherein the administration occurs ex vivo.

11. The process of claim 9 wherein the administration occurs in vivo.

12. A pharmaceutical composition for administration to a mammal suffering from a disorder involving an inadequate immune response to a cancer, viral or bacterial infection, said composition comprising an effective amount of an agent that can augment the immune response of said mammal, said agent comprising, as the sole immune response enhancing agent, an agent that can augment the level of tapasin in target cells of the mammal, and a suitable adjuvant or carrier.

13. The pharmaceutical composition of claim 12 wherein the disorder is selected from the group consisting of cervical cancer, colorectal cancer, non-Hodgkin lymphoma, lymphoma, stomach carcinoma, liver carcinoma, leukemia, kidney carcinoma, pancreatic carcinoma, sarcoma, mesothelioma, uterine carcinoma, bladder carcinoma, head and neck carcinoma, esophageal cancer, testicular cancer, ovarian carcinoma, thyroid cancer, oral cancer, stomach cancer, cancer of the larynx, Hodgkin lymphoma, breast carcinoma, prostate carcinoma, melanoma, non-melanoma skin cancer, basal cell skin cancer, squamous cell skin cancer, lung carcinoma brain cancer, multiple myeloma, influenza, small pox, and tuberculosis.

14. A process of enhancing an immune response to an antigen comprising administering to a cell or animal in need thereof, in combination, as the sole immune response enhancing agents, an effective amount of (a) an agent that can augment the level of tapasin in a target cell bearing the antigen, and (b) an agent that can augment the level of TAP-1 in said target cell.

15. The process of claim 14 wherein said agents comprise nucleic acids encoding tapasin and TAP-1, respectively.

16. The process of claim 14 wherein said agents comprise one or more viral vectors containing nucleic acids encoding tapasin and TAP-1, respectively.

17. The process of claim 16 wherein the viral vectors are adenoviral vectors.

18. The process of claim 14 wherein said agents comprise one or more plasmid vectors containing nucleic acids encoding tapasin and TAP-1, respectively.

19. The process of claim 14 wherein the target cell is a tumor cell.

20. The process of claim 14 wherein the target cell is a virally infected cell or a bacterial cell.

21. The process of claim 14 wherein said agents comprises tapasin and TAP-1, respectively.

22. The process of claim 14 wherein the animal is a human patient.

23. The process of claim 22 wherein the administration occurs ex vivo.

24. The process of claim 22 wherein the administration occurs in vivo.

25. A pharmaceutical composition for administration to a mammal suffering from a disorder involving an inadequate immune response to a cancer, viral or bacterial infection, said composition comprising an effective amount of agents that can augment the immune response of said mammal, said agents comprising, in combination, as the sole immune response enhancing agents (a) an agent that can augment the level of tapasin in target cells of the mammal, and (b) an agent that can augment the level of TAP-1 in target cells of the mammal, and a suitable adjuvant or carrier.

26. The pharmaceutical composition of claim 25 wherein the disorder is selected from the group consisting of cervical cancer, colorectal cancer, non-Hodgkin lymphoma, lymphoma, stomach carcinoma, liver carcinoma, leukemia, kidney carcinoma, pancreatic carcinoma, sarcoma, mesothelioma, uterine carcinoma, bladder carcinoma, head and neck carcinoma, esophageal cancer, testicular cancer, ovarian carcinoma, thyroid cancer, oral cancer, stomach cancer, cancer of the larynx, Hodgkin lymphoma, breast carcinoma, prostate carcinoma, melanoma, non-melanoma skin cancer, basal cell skin cancer, squamous cell skin cancer, lung carcinoma brain cancer, multiple myeloma, influenza, small pox and tuberculosis.
